(19) United States
(12) Patent Application Publication
(10) Pub. No.: US 2025/0336470 A1
(43) Pub. Date: Oct. 30, 2025

(54) SYSTEM AND METHOD FOR USING GRAPH NEURAL NETWORK ARCHITECTURE FOR PREDICTING DRUG-TARGET INTERACTIONS

(71) Applicant: Edammo, Inc., Iowa City, IA (US)

(72) Inventors: Edward Ratner, Stirling, NJ (US); Christopher J. Douglas, Coralville, IA (US)

(21) Appl. No.: 19/190,417

(22) Filed: Apr. 25, 2025

Related U.S. Application Data
(60) Provisional application No. 63/638,648, filed on Apr. 25, 2024.

(51) Int. Cl. G16B 15/30; G06N 3/09; G16B 40/20

(52) U.S. Cl. CPC G16B 15/30; G06N 3/09; G16B 40/20

(57) ABSTRACT

An apparatus, system and method are disclosed for predicting a graph edge of a graph neural network architecture. A form of classification is performed in which node feature samples are evaluated for different ensembles, such as a L₀₀ ensemble and an L_{0.1} ensemble. The technique can be used to identify candidate drug target interactions.

### CROSS REFERENCE TO RELATED APPLICATION

The present application claims the benefit of U.S. Provisional Application No. 63/638,648, which was filed on Apr. 25, 2024.

### INCORPORATION BY REFERENCE

The following patent applications by one or more of the inventors of the present application are hereby incorporated by reference: U.S. patent application Ser. No. 17/503,490 filed on Oct. 18, 2021, entitled “System and Method for Dynamic Model Training with Human in the Loop”; and U.S. patent application Ser. No. 18/945,135, filed on Nov. 12, 2024, titled “System and Method for Ensembling Learners with Highly Variable Class-Based Performance”,

The academic papers listed in this application are also hereby incorporated by reference.

### FIELD OF THE INVENTION

The present disclosure generally relates to graph neural networks used to implement models used for different applications, including advanced drug development. M ore particularly, the present disclosure is related to graph neural network architecture using ensembles of shallow stochastic machine learning models, such as ensembles of Extreme Learning Machines, to perform edge prediction for a variety of purposes, including predicting drug target interactions.

### BACKGROUND

There are numerous tradeoffs between different machine learning technologies. This has limited the ability to use machine learning architectures in certain types of end-use applications, such as predicting drug-target interactions (DTIs).

The development of new molecular entities (NM E) takes an average of 9 to 12 years [1] and costs about 1.8 billion dollars [See, e.g., Paul, S. M.; Mytelka, D. S.; Dunwiddie, C. T.; Persinger, C. C.; M unos, B. H.; Lindborg, S. R.; Schacht, A. L. How to improve R & D productivity: The pharmaceutical industry's grand challenge. Nat Rev. Drug Discov. 2010, 9, 203-214]. Additionally, the US Food and Drug Administration (FDA) approves only 20 NM Es per year on average [See, e.g., R. Chen, X. Liu, S. Jun, J. Lin, and J. Liu, “Machine learning for drug-target interaction prediction,” Molecules, vol. 23, no. 9, pp. 2208, 2018]. The substantial financial, time, and resource investments required have prompted researchers to develop innovative computational approaches aimed at optimizing the drug development process. Predicting drug-target interactions (DTIs) remains a key challenge in this process [See, e.g., J. Knowles and G. Gromo, “Target selection in drug discovery,” Nature Reviews Drug Discovery, 2(1): 63-69, 2003]. Not only do DTIs inform new applications of existing drugs (i.e., drug repositioning), but they also may provide insights into developing new drugs using virtual screening.

Various computational approaches have been proposed for identifying molecular similarities. These models are often converted to semi-supervised learning tasks. Link prediction methods structure knowledge graphs where various biomedical entities serve as nodes and their connections and interactions as edges. Link prediction methods have taken a variety of forms, including node similarity prediction using local topological characteristics, label propagation probability soft logic and graph traversal algorithms. Graph-embedding approaches transform graphs into lower-dimensional space using embedding layers, which preserve much of the structural network information.

Most DTI computational approaches may be classified as either ligand-based, molecular docking, chemogenomic, or feature learning approaches. M olecular docking methods work to predict the 3D structures of drug-target pairs using scoring functions. Y amanishi et al. [See Y. Y amanishi, M. Araki, A. Gutteridge, W. Honda, and M. Kanehisa, M., “Prediction of drug-target interaction networks from the integration of chemical and genomic spaces,” Bioinformatics, vol. 24, no. 13, pp. i232-i240, 2008.] were among the first to illustrate the predictive power of docking approaches, utilizing a combination of chemical and genomic spaces as input to a kernel regression model. To overcome the massive computational complexity of this approach, Bleakley [See, e.g., K. Bleakley and Y. Y amanishi, “Supervised prediction of drug-target interactions using bipartite local models,” Bioinformatics, vol. 25, no. 18, pp. 2397-2403, 2009] proposed the use of local, as opposed to global, bipartite models and attained greater prediction power and lower computational complexity than the former approach.

Additionally, regularized matrix factorization methods were proposed, which multiply latent compound and target matrices to predict DTIs. These approaches proved more successful than previous approaches but were eventually abandoned because similarity-based methods were only useful within specific protein classes. Additionally, docking approaches require knowledge of the crystallized structure of proteins which is often not available. Feature learning approaches use mappings of DTIs into numerical representations to learn potential relationships of unknown drug-target pairs in a binary classification setting. In feature-based approaches, molecular fingerprints are often used to represent the substructure of a compound [See, e.g., S. Wang, D. Liu, M. Ding, Z. Du, Y. Zhong, T. Song, J. Zhu, and R. Zhao, “Se-onionnet: A convolution neural network for protein-ligand binding affinity prediction,” Frontiers in Genetics, vol. 11, pp. 607824, 2021].

Additionally, composition, transition, and distribution (CTD) are often used to represent proteins. Initially, feature-based methods using these representations performed worse than previously proposed quantitative structure-activity relationship (QSAR) models. Improvements were made using minwise hashing and interactome networks. Despite these improvements, traditional machine learning models struggled to leverage these representations. In recent years, more complex machine learning approaches, such as Deep Learning, have been proposed in hopes of overcoming the limitations of traditional machine learning models.

Various machine learning architectures have been proposed for the prediction of DTIs, including Support Vector Machines (SV M s), Random Forests, and Convolutional Neural Networks (CNNs) and Deep Learning. Many of these approaches utilize simplified molecular-input line entry system (SMILES) and amino acid sequences as features in the model. While traditional machine learning models often lose valuable information of these features during transformation, more complex models can better retain local residue patterns.

### SUMMARY

An apparatus, system, and method are disclosed for using Graph Shallow Random Neural Networks (GSRNN) for predicting drug target interactions based on predictions of candidate graph edges. However, the approach has more general applicability to other problems.

An example implementation of uses features extracted from drug, protein, and graph structures. An example method of predicting a drug target interaction (DTI) from a candidate edge of a graph neural network, includes accessing a training data set and a validation data set for a knowledge graph, the training data set including approved drug target interactions having drug, protein, and graph features; generating a first ensemble of shallow random networks to contain drug node graph features, protein node graph features, drug structure features, and protein structure features for neighboring nodes directly connected to a candidate edge of the knowledge graph; and generating at least one additional ensemble of shallow random networks for nodes that include at least some features contributed from next nearest neighbor; for each ensemble, generating a prediction for the edge under consideration; and averaging the predictions to generate an average prediction whether the candidate edge is a candidate for drug target interactions.

### BRIEF DESCRIPTION OF THE DRAWINGS

The present disclosure is illustrated by way of example, and not by way of limitation, in the figures of the accompanying drawings in which like reference numerals are used to refer to similar elements.

FIG. 1 is a block diagram illustrating a general system using an ensemble of machine learning models in accordance with an implementation.

FIG. 2 illustrates a server-based example of a system to use an ensemble of ELMs to implement a model in accordance with an implementation.

FIG. 3 is a high-level flowchart of an example method in accordance with an implementation.

FIG. 4 is a high level L_{0,0} ensemble figure of a feature creation process in accordance with an implementation.

FIG. 5 is a high level L_{0,1} ensemble figure of a feature creation process in accordance with an implementation.

FIG. 6 is a high level L_{0,0} ensemble figure of a feature creation process for DTI in accordance with an implementation.

FIG. 7 is a high level L_{0,1} ensemble figure of a feature creation process for DTI in accordance with an implementation.

### DETAILED DESCRIPTION

A knowledge graph and neural machine learning techniques to make edge predictions classifying edges based on sample features for various applications, including predicting drug target interactions. A graph-based neural network architecture approach for predicting DTIs, which can be referred to as Graph Shallow Random Neural Networks (GSRNN). For predicting DTIs based on evaluating graph edges, an implementation of this approach uses features extracted from drug, protein, and graph structures. There are various machine learning M L architectures that use randomly generated weights, including radial basis function networks (RBFN), random vector functional link networks (RVFL), Schmidt's method [See, e.g., W. F. Schmidt, M. A. Kraaijveld, R. P. Duin, “Feed forward neural networks with random weights,” In International conference on pattern recognition, 1-1, 1992], and Extreme Learning Machines (ELMs) [See, e.g., G. B. Huang, Q.-Y. Zhu, C.-K. Sie, “Extreme learning machine: theory and applications,” Neurocomputing, 70(1-3):489-501, 2006]. Each of these architectures operates under Rosenblatt's assumption [See, e.g., F. Rosenblatt, “The perceptron: a probabilistic model for information storage and organization in the brain,” Psychological review, vol. 65, no. 6, pp. 386, 1958 that randomly connected units can give specific responses to certain stimuli under a given set of constraints]. Among these architectures, EL M s have received the most attention due to the rigorous theoretical proofs which suggest that EL M s have universal approximation capabilities. In the case of ELMs, almost all nonlinear piecewise continuous functions can be used as random hidden nodes, including Sigmoid, wavelets, Fourier series and biological neurons. ELMs have been shown to outperform classical and Deep Learning architectures in various scenarios [See B. Warner, E. Ratner, and A. Lendasse, “Edammo's extreme automl technology-benchmarks and analysis,” in International Conference on Extreme Learning Machine, 2021. 152-163; E. Ratner, E. Farmer, B. Warner, C. Douglas, and A. Lendasse, “Extreme AutoML: Analysis of Classification, Regression, and NLP Performance. 2024,” arXiv preprint arXiv: 2412.07000v]; see also A. Iftikhar, M. Basheri, M. J. Iqbal, and A. Rahim, “Performance comparison of support vector machine, random forest, and extreme learning machine for intrusion detection,” IEEE access 6, pp. 33789-33795, 2018].

In one implementation, graph-based ELMs are used for the graph-based neural network architecture.

In one implementation, a combination of global and local features is used for each node under consideration. In one implementation, the features are computed on a training network once for each node and then used to create each edge sample feature set in both training and testing. Each node will normally contribute its features to multiple edges in a knowledge graph topology.

In one example implementation, the global feature is based on computing the node centrality [See, e.g., T. Opsahl, F. Agneessens, J. Skvoretz, “Node centrality in weighted networks: Generalizing degree and shortest paths,” Social Networks, vol. 32, no. 3, pp. 245-251, 2010]. In one example implementation, the local features include the node degree, the entropy of its first-degree neighborhood, and the random walk probabilities of its first-degree neighbors [See e.g. L. Backstrom and J. Leskovec, “Supervised random walks: predicting and recommending links in social networks, in Proceedings of the fourth ACM international conference on Web search and data mining, 2011, pp. 635-644]. In one implementation, the entropy of each node is computed, treating it and its first-degree neighbors as a self-contained network [Sce, e.g., G. Simonyi, “Perfect graphs and graph entropy. An updated survey,” Perfect graphs, 293-328, 2001; E. Csoka, V. Harangi, and B. Virag, “Entropy and expansion,” pp. 2428-2444, 2020].

In the case of random walk features, the node is considered as the origin and the probability computed of each neighbor node being the location after a 5-step random walk. This provides a local characterization of the network up to the depth of approximately three. In one implementation, 16 neighbor random walk features are used. For example, the node centrality and the random walk probabilities may be computed using algorithms like SNAP [See, e.g., J. Leskovec and R. Sosi c, “Snap: A general-purpose network analysis and graph-mining library,” In ACM Transactions on Intelligent Systems and Technology (TIST) 8.1, 2016].

For a DTI implementation, drug features need to be selected. This may include generating features from drug databases. In one implementation, drug features were generated using the PubChem [See, e.g., S. Kim, S. and E. E. Bolton, E. E., “PubChem: A Large-Scale Public Chemical Database for Drug Discovery,” Open Access Databases and Datasets for Drug Discovery, 39-66, 2024] and the embedding approach in the DeepPurpose library [See, e.g., K. Huang, T. Fu, L. M. Glass, M. Zitnik, C. Xiano, and J. Sun, “Deep-purpose: a deep learning library for drug-target interaction prediction,” Bioinformatics, vol. 36, no. 22-23, pp. 5545-5547, 2020.]

This approach can be adapted to tap into the large-scale chemical database PubChem to create embeddings. Additionally, in one implementation M organ fingerprints [See, e.g., Y. Ding, M. Chen, C. Guo, P. Zhang, and J. Wang, “Molecular fingerprint-based machine learning assisted QSA R model development for prediction of ionic liquid properties,” Journal of Molecular Liquids, vol. 326, pp. 115212, 2021] from the DeepPurpose library were selected Drug features can be built, for example, using tools such as TransformerCPI2.0 [See, e.g., L. Chen, Z. Fan, J. Chang, R. Y ang, H. Hou, H. Guo, . . . and M. Zheng, “Sequence-based drug design as a concept in computational drug design,” Nature Communications, vol. 14, no. 1, pp. 4217, 2023].

In one implementation, features were extracted after the application of a pre-trained graph convolutional network layer (GCN), which can be accessed in their repository. Finally, in one implementation features were created using the DescriptaStorus repository.

In one implementation, a novel neural network architecture for graph modeling consists of ensembles of shallow random networks implemented as ELMs. As ELMs can be rapidly trained, the ELM ensemble can be trained in parallel when a search query is initiated.

The problem of edge prediction on a graph in one implementation is performed as a binary classification task. The shallow networks consist of one hidden layer and one input layer where the weights are set randomly. These types of networks have been known by various names in literature and include ELMs. A review of ELM technology is provided in the article by Mustafa A bbas A bbod Albadr et al, “Extreme Learning Machine: A Review”, International Journal of Applied Engineering Research, ISSN 0973-4562 Vol. 12, No. 14 (217), pp 4610-4623, the contents of which are hereby incorporated by reference. Additional background on ELMs on determining weights is found in a paper by G. B. Huang, et al, “Extreme Learning Machine: Theory and Applications,” Neurocomputing, 70(10:489-501 (2006), the contents of which are hereby incorporated by reference.

In one implementation, a dynamic, parameter-free methodology to create ensembles uses techniques similar to those described in the following references incorporated by reference: B. Warner, E. Ratner, and A. Lendasse, “Edammo's extreme automl technology-benchmarks and analysis,” in International Conference on Extreme Learning Machine, 2021. 152-163; E. Ratner, E. Farmer, B. Warner,C. Douglas, and A. Lendasse, “Extreme AutoML: Analysis of Classification, Regression, and NLP Performance. 2024,” arXiv preprint arXiv: 2412.07000; B. Warner, E. Ratner, K. Carlous-Khan, C. Douglas, and A. Lendasse, “Ensemble Learning with Highly Variable Class-Based Performance,” Machine Learning and Knowledge Extraction, vol. 6, number 4, pp. 2149-2160, 2024].

In an ensemble paradigm, the training data is split into model training data and validation data, which is used to determine weights for the ensemble. In one implementation, a dynamic ensemble of shallow networks is created for nodes at each depth from the edge under consideration. This includes ensembles including feature contributions from neighboring nodes directly connected to an edge and ensembles that include feature contributed from next nearest neighbors. These ensembles are themselves ensembled for final prediction as described below in more detail.

In one implementation, a definition of an edge prediction model L (i,j) for model L (i,j) uses the features i+j+2 nodes to predict the edge. The features come from the left node, left node nearest neighbor, left node next nearest neighbor up left node ith nearest neighbor and also from the right node, right node nearest neighbor up to right nodes jth nearest neighbor. For a given sample, the neighbor at the depth of k+1 has to be a neighbor of the node at depth k. Thus, for any sample the features are collected from nodes along a path from the ith nearest neighbor of the left node to jth nearest neighbor of the right node. Each unique path provides a unique sample.

For training all unique combinations of neighbor nodes contribute a sample to the training set. For prediction, each valid combination of neighbors for a candidate edge yields a prediction and the final prediction is the average of these predictions.

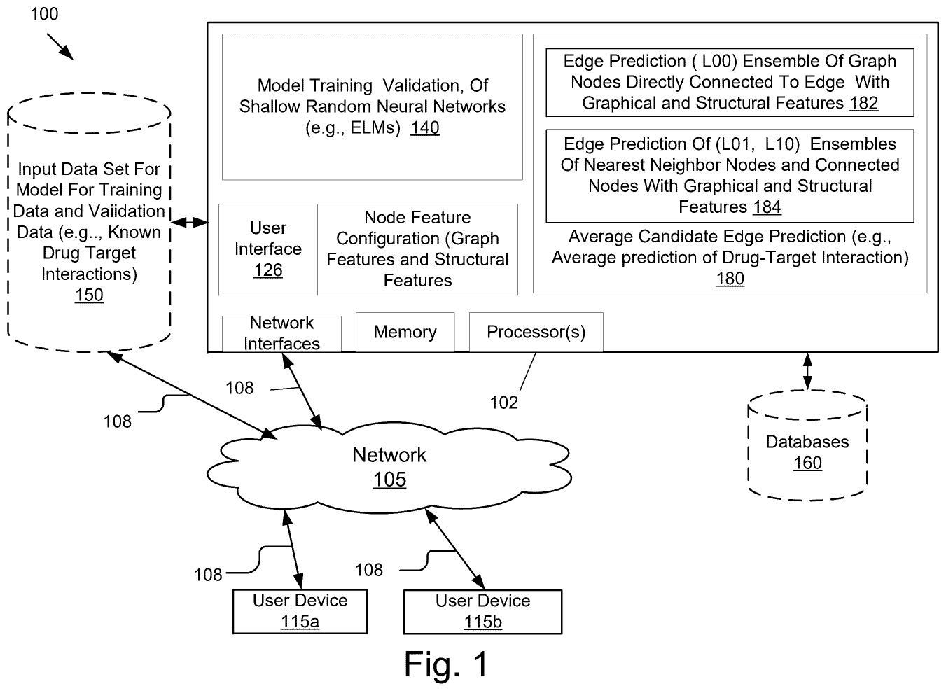

FIG. 1 illustrates a general system for performing edge prediction of a knowledge graph, where for example, the edge prediction may correspond to a DTI prediction. A general system may, for example, be supported by a computer server 102 having a network interface, memory, and processor. Interfaces 108 are supported to, for example, access an input data set for training and validation 150, which, for example, may be a database of known drug target interactions. More generally, different input data sets may be provided should, for example, some ensembles perform better for some data sets than others. Model training and validation 140 is provided of the shallow random neural networks, which may, for example, be implemented in ELMs. A user interface 126 may be provided. A configuration of local and global node features and other structural feature 128 may be supported. In one implementation, a candidate edge prediction 180 is based on an average of the edge predictions for different ensembles 182 and 184 generated at different depths, as discussed below in more detail. The overall system can be used in many different ways, but predicting edges corresponding to predicting DTIs is one implementation.

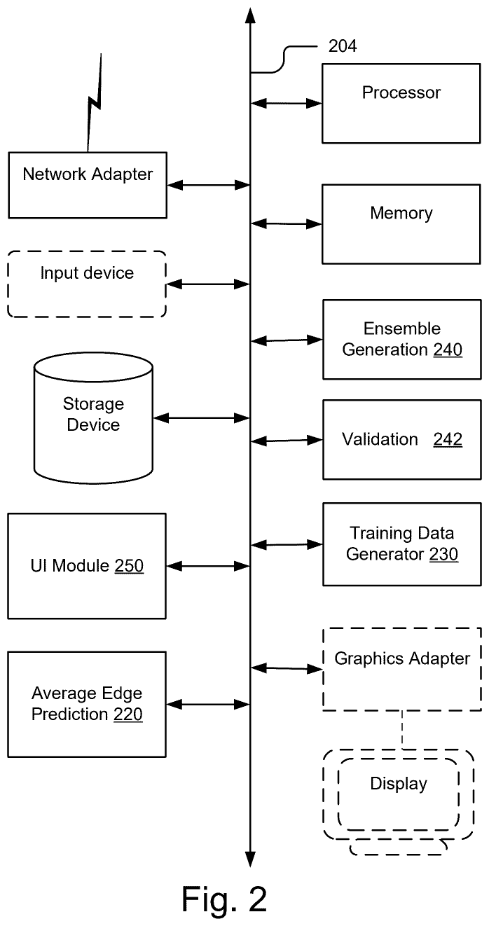

FIG. 2 illustrates a general server implementation having a processor, memory, an adapter input device, storage device, and other components coupled by a communication bus 204. A component, such as a memory, may be used to story code for ensemble generation 240, training data generation 230, validation 242, a UI module 250, and average edge prediction 220.

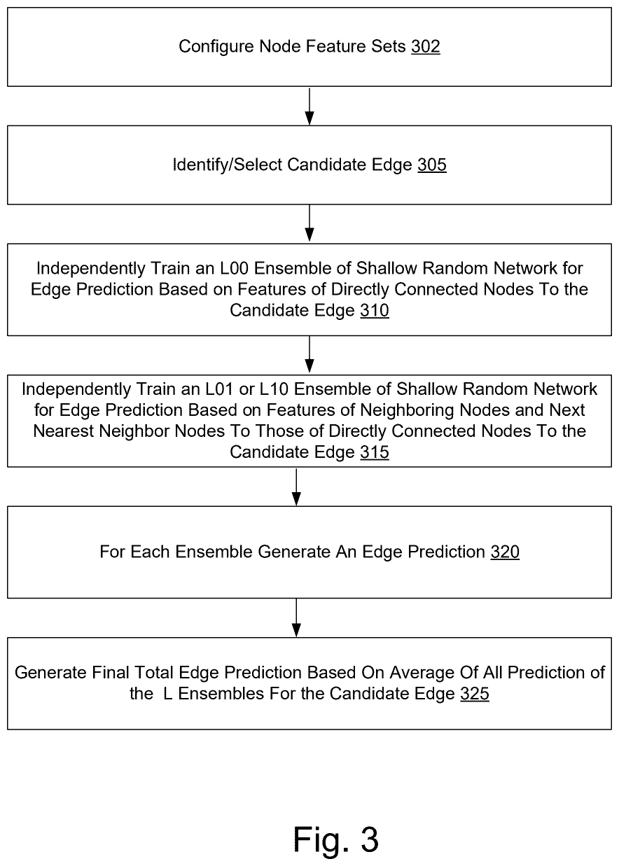

FIG. 3 is a high-level flowchart of aspects of a method in accordance with an implementation. In one implementation, in block 302, node feature sets are configured. In block 305 a candidate edge is selected. As discussed below in more detail, an ensemble (L₀₀) is trained in block 310 based on features of directly connected nodes. Additionally, other ensembles are trained in block 315 to account for features of next nearest neighbors, such as a L_{0,1} ensemble. In block 320, edge predictions are generated for each ensemble. In block 325, an average edge prediction is generated based on all the edge predictions from the different ensembles.

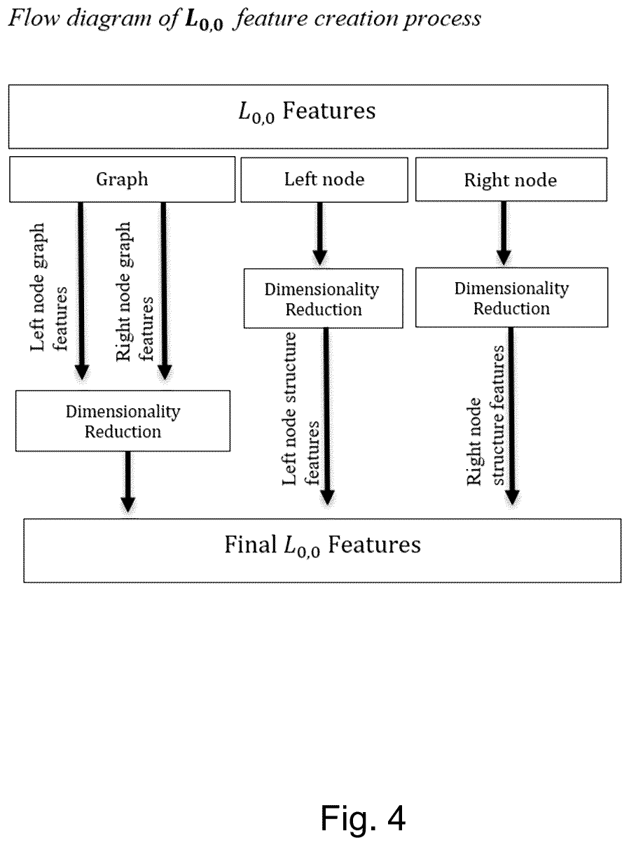

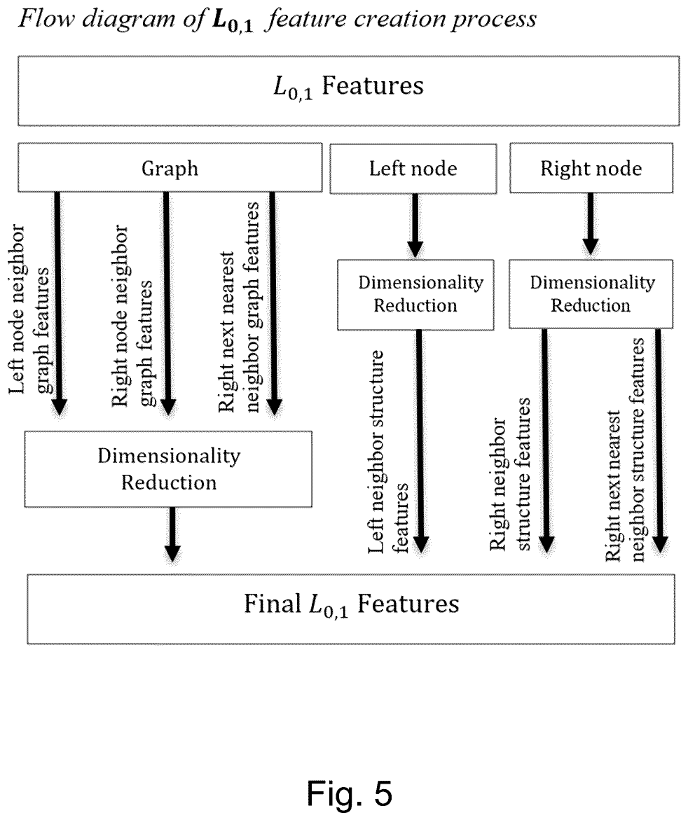

As an illustrative example, consider the general example of FIGS. 4 and 5 regarding flow diagrams of feature creation process. In FIG. 4, the feature creation process starts with ensemble L_{0,0} which contains the features of the nodes that the edge under consideration connects. There are left node graph features and right node graph features. There are left node structural features and right node structural features. Dimensionality Reduction is used independently for each feature types.

The dimensionality reduction may include a variety of different types known in the art. Principal component analysis (PCA)—“simple” linear method that tries to preserve global distances; Multidimensional scaling (MDS)-tries to preserve global distances; Sammon's projection—a variation of the MDS, pays more attention to short distances; Isometric mapping of data manifolds (ISOMAP)—a graph-based method (of the MDS spirit); Uniform Manifold Approximation and Projection (UMAP)—similar to ISOM AP, but more robust; Curvilinear component analysis (CCA)—MDS-like method that tries to preserve distances in small neighborhoods; Maximum variance unfolding—maximizes variance with the constraint that the short distances are preserved (an exercise in semidefinite programming); Self-organizing map (SOM)—a flexible and scalable method that tries a surface that passes through all data points (originally developed at HUT); Laplacian eigenmap-when only connections are known; independent component analysis (ICA)—a fast linear method, suitable for some applications; and Nerv (N Eighbor Retrieval Visualizer, originally developed at HUT). The result of this portion of the feature creation process is the final L_{0,0} features.

Referring to FIG. 5, in one implementation process includes exploring the L_{0,1} ensemble and its feature creation process, which contains the features from the left node, the right node and the first-degree neighbor of the right node feature creation. Thus, in this example, the final L_{0,1} features include the left node neighbor graph features, the right node graph features, the right next nearest neighbor graph features, the left neighbor structure features, the right neighbor structure features, and the right next nearest neighbor structure features.

Additional ensembles may be explored such as the L_{1,0} ensembles, which uses the features from the left node, the right node, and one first degree neighbor of the left node. Moreover, the number of L ensembles can in theory be selected to whatever depth is desired, such as exploring contributions from next to next nearest neighbors.

In one implementation, each L ensemble is trained independently and the viable sample for each ensemble used to predict the outcome for the candidate edge. For instance, for the L_{0,1} ensemble each neighbor of the right node (e.g., a protein node in FIG. 8) would yield a feature sample. A prediction is made using each of those samples and the result is averaged for the final L_{0,1} ensemble prediction. Equation 1 below illustrates this process.

$${L}_{0 , 1} ( {e}_{i , j} ) = \frac{{\sum }_{k ∈ {neighbor}_{j , k \neq i}} {L}_{0 , 1}^{f} ( i , j , k )}{J - 1} \qquad ( 1 )$$

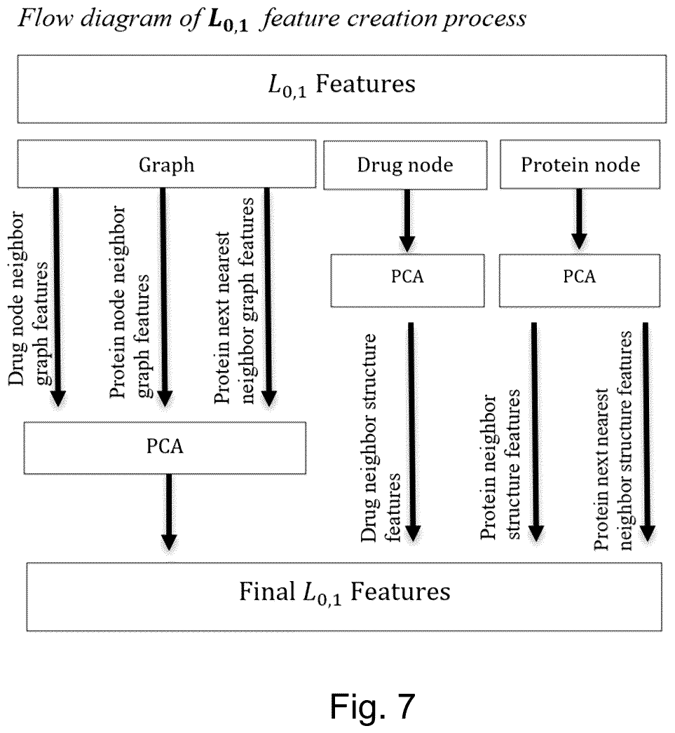

Here, e denotes the edge under consideration. Then i is the left node (e.g., the drug in FIG. 7), j is the right node (e.g., the protein in FIG. 7, and L^{f} denotes the model using the features from the nodes in the argument. J is the degree of node j.

The final total prediction is the average over all the L ensembles for the candidate edge in question. Equation 2 shows the relevant formula.

$$L ( e ) = \frac{{\sum }_{all L} {L}_{m , n} ( e )}{N} \qquad ( 2 )$$

Here, N is the total number of L ensembles in the model.

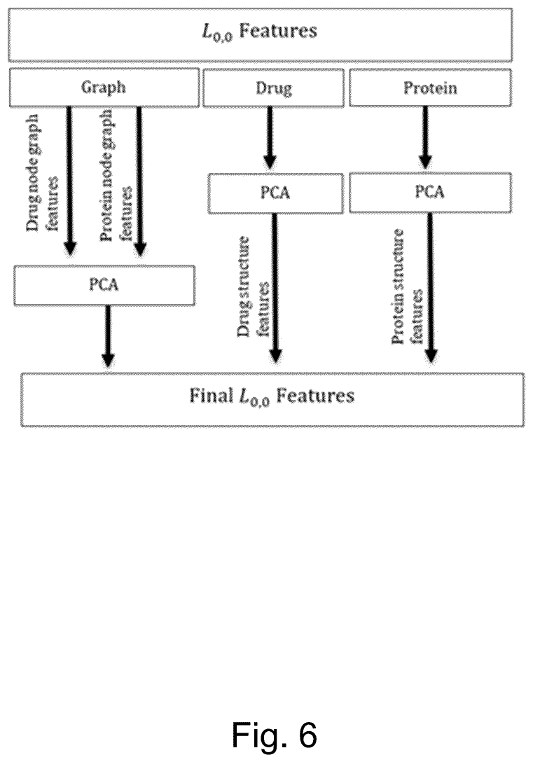

FIGS. 6 and 7 visualize the feature creation process for DTIs. PCA is illustrated as being an example of dimensionality reduction, although a wide variety of different types of dimensionality reduction techniques may be used as previously discussed. In the example, of FIG. 6, the final L_{0,0} ensemble includes drug node graph features, protein node graph features, drug structure features, and protein structure features. FIG. 7 shows how the final L_{0,1} features include drug node neighbor graph features, protein node neighbor graph features, protein next nearest neighbor graph features, drug neighbor structure features, protein neighbor structure features, and protein next nearest neighbor structure features.

### DTI Examples

In the examples of FIGS. 6-7, edge prediction process makes predictions regarding potential DTIs. It thus provides extremely useful information for researchers.

A set of initial preliminary experiments by the inventors was performed in an initial benchmarking of the performance of a variety of drug, protein, and graph feature configurations. After creating features, a principal component analysis (PCA) was run on each feature set, testing a variety of principal components. For each of the experiments, 10 Monte Carlo simulations were run, using 90% of the edge data for training and validation and 10% for testing. Node features were building the networks consisting of only training edges to prevent data leakage. The performance of the models was evaluated for accuracy and F1 score.

DTI Baselines were considered by comparing performance with several examples of previous state-of-the-art methods including M PNN-CNN, DeepConv, TransformerCPI, M oIT rans, Morgan-CNN, and DeepFusion. The summary results of the benchmarking is shown in table I below. We include our top two configurations in this table.

TABLE I
RESULTS SUMMARY

DTI Prediction Results

| | | | F1 |
|---|---|---|---|
| | Method | Accuracy | Score |
| 1 | Morgan-CNN | 0.800 | 0.795 |
| 2 | MPNN-CNN | 0.798 | 0.795 |
| 3 | DeepConv-DTI | 0.803 | 0.796 |
| 4 | TransformerCPI | 0.803 | 0.796 |
| 5 | MolTrans | 0.795 | 0.802 |
| 6 | DeepFusion | 0.830 | 0.827 |
| 7 | SRNN-1 (based on this | 0.900 | 0.900 |
| | disclosure) | | |

The results outperform SOTA methodologies, as reported in [See T. Song, X. Zhang, M. Ding, A. Rodriguez-Paton, S. Wang and G. Wang, “DeepFusion: A deep learning based multi-scale feature fusion method for predicting drug-target interactions,” Methods, vol. 204, pp. 269-277, 2022].

Consider now various ensemble models as follows.

SRNN-1: We produced many ensemble models that outperformed previous SOTA metrics. SRNN_1 achieved the best performance. In this ensemble, the L_{0,0} model used PubChem for the drug embedding and TapeBERT [See, e.g., R. Rao, N. B hattacharya, N. Thomas, Y. Duan, P. Chen, J. Canny, . . . and Y. Song, “Evaluating protein transfer learning with TAPE. 32,” Advances in neural information processing systems, Vol. 32, 2019] for protein embedding. The post-PCA complexion of this model was comprised of thirteen graph features, three drug features, and three protein features. Interestingly, the L_{0,1} model was solely comprised of ten graph features (i.e., no protein or drug features).

SRNN_2: In our second-best ensemble, L_{0,0} model used PubChem (in DeepPurpose library) for drug embedding and conjoint triad for protein embedding. After PCA, we used nine graph features, three drug features, and three protein features. For the L_{0,1} model, we only used 10 graph features.

SRNN-3: In this ensemble, the L_{0,0} model used DescriptaStorus [52] for drug embedding and TapeBERT for protein embedding. After PCA, we used thirteen graph features, two drug features, and two protein features. For the L_{0,1} model, we used only 10 novel graph feature PCA components.

SRNN-4: In this ensemble, the L_{0,0} model used Descriptastorus [52] for drug embedding and TapeBERT for protein embedding. After PCA, we used thirteen graph features, two drug features, and two protein features. For the L_{0,1} model, we used DescriptaStorus for drug embedding, TapeBERT for protein embedding, and Descriptastorus for neighbor embedding. After PCA, we used two components for each of these sets. This model also used ten novel graph feature PCA components.

SRNN-5: In the SRNN-5 ensemble, the L_{0,0} model used PubChem for drug embedding and conjoint triad for protein embedding. After PCA, we used three drug and protein features. This model also used nine novel graph features. For the L_{0,1} model, we used Descriptastorus for drug and neighbor embedding and TapeBERT for protein embedding. After PCA, we used two components for each of these sets. This model also used ten novel graph feature PCA components.

SRNN-G: We also ran an ensemble of only graph features. This provides insights into the predictive impact of various protein and drug embedding strategies. This ensemble, SRNN-G, used ten PCA component graph features for each model in the ensemble. Notably, this purely graph-based ensemble attained a better F1 score (0.8913) and accuracy (0.8913) than previous SOTA approaches (see table 1). Table II, below, presents our top six ensembles in our initial experiments according to F1 score and accuracy.

TABLE II
NOVEL ENSEMBLE RESULTS

DTI Prediction Results

| | | | F1 |
|---|---|---|---|
| | Model | Accuracy | Score |
| 1 | SRNN-1 | 0.8998 | 0.8999 |
| 2 | SRNN-2 | 0.8968 | 0.8961 |
| 3 | SRNN-3 | 0.8963 | 0.8969 |
| 4 | SRNN-4 | 0.8955 | 0.8951 |
| 5 | SRNN-5 | 0.8953 | 0.8948 |
| 7 | SRNN-G | 0.8916 | 0.8913 |

Initial experiments illustrate that our novel graph features and neural network architecture enrich model quality and boost predictive performance significantly. These experiments also shed light on the most accurate drug and protein embedding methods when combined with our novel graph features.

### Alternate Implementations

In the above description, for purposes of explanation, numerous specific details were set forth. It will be apparent, however, that the disclosed technologies can be practiced without any given subset of these specific details. In other instances, structures and devices are shown in block diagram form. For example, the disclosed technologies are described in some implementations above with reference to user interfaces and particular hardware.

Reference in the specification to “one embodiment”, “some embodiments” or “an embodiment” means that a particular feature, structure, or characteristic described in connection with the embodiment is included in at least some embodiments of the disclosed technologies. The appearances of the phrase “in some embodiments” in various places in the specification are not necessarily all referring to the same embodiment.

Some portions of the detailed descriptions above were presented in terms of processes and symbolic representations of operations on data bits within a computer memory. A process can generally be considered a self-consistent sequence of steps leading to a result. The steps may involve physical manipulations of physical quantities. These quantities take the form of electrical or magnetic signals capable of being stored, transferred, combined, compared, and otherwise manipulated. These signals may be referred to as being in the form of bits, values, elements, symbols, characters, terms, numbers, or the like.

These and similar terms can be associated with the appropriate physical quantities and can be considered labels applied to these quantities. Unless specifically stated otherwise as apparent from the prior discussion, it is appreciated that throughout the description, discussions utilizing terms, for example, “processing” or “computing” or “calculating” or “determining” or “displaying” or the like, may refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The disclosed technologies may also relate to an apparatus for performing the operations herein. This apparatus may be specially constructed for the required purposes, or it may include a general-purpose computer selectively activated or reconfigured by a computer program stored in the computer.

The disclosed technologies can take the form of an entirely hardware implementation, an entirely software implementation or an implementation containing both software and hardware elements. In some implementations, the technology is implemented in software, which includes, but is not limited to, firmware, resident software, microcode, etc.

Furthermore, the disclosed technologies can take the form of a computer program product accessible from a non-transitory computer-usable or computer-readable medium providing program code for use by or in connection with a computer or any instruction execution system. For the purposes of this description, a computer-usable or computer-readable medium can be any apparatus that can contain, store, communicate, propagate, or transport the program for use by or in connection with the instruction execution system, apparatus, or device.

A computing system or data processing system suitable for storing and/or executing program code will include at least one processor (e.g., a hardware processor) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including, but not limited to, keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modems, and Ethernet cards are just a few of the currently available types of network adapters.

Finally, the processes and displays presented herein may not be inherently related to any particular computer or other apparatus. Various general-purpose systems may be used with programs in accordance with the teachings herein, or it may prove convenient to construct a more specialized apparatus to perform the required method steps. The required structure for a variety of these systems will appear from the description below. In addition, the disclosed technologies were not described with reference to any particular programming language. It will be appreciated that a variety of programming languages may be used to implement the teachings of the technologies as described herein.

The foregoing description of the implementations of the present techniques and technologies has been presented for the purposes of illustration and description. It is not intended to be exhaustive or to limit the present techniques and technologies to the precise form disclosed. M any modifications and variations are possible in light of the above teaching. It is intended that the scope of the present techniques and technologies be limited not by this detailed description. The present techniques and technologies may be implemented in other specific forms without departing from the spirit or essential characteristics thereof. Likewise, the particular naming and division of the modules, routines, features, attributes, methodologies, and other aspects are not mandatory or significant, and the mechanisms that implement the present techniques and technologies or its features may have different names, divisions, and/or formats. Furthermore, the modules, routines, features, attributes, methodologies, and other aspects of the present technology can be implemented as software, hardware, firmware, or any combination of the three. Also, wherever a component, an example of which is a module, is implemented as software, the component can be implemented as a standalone program, as part of a larger program, as a plurality of separate programs, as a statically or dynamically linked library, as a kernel loadable module, as a device driver, and/or in every and any other way known now or in the future in computer programming. Additionally, the present techniques and technologies are in no way limited to implementation in any specific programming language, or for any specific operating system or environment. Accordingly, the disclosure of the present techniques and technologies is intended to be illustrative, but not limiting.

What is claimed is:

1. A method of predicting a drug target interaction (DTI) from a candidate edge of a graph neural network, comprising:
accessing a training data set and a validation data set for a knowledge graph, the training data set including approved drug target interactions having drug, protein, and graph features;
generating a first ensemble of shallow random networks to contain drug node graph features, protein node graph features, drug structure features, and protein structure features for neighboring nodes directly connected to a candidate edge of the knowledge graph; and
generating at least one additional ensemble of shallow random networks for nodes that include at least some features contributed from next nearest neighbors;
for each ensemble, generating a prediction for the edge under consideration; and
averaging the predictions to generate an average prediction whether the candidate edge is a candidate for drug target interactions.

2. The method of claim 1, wherein the at least one additional ensemble contains features from drug graph features, protein node graph features, protein next nearest neighbor graph features, drug neighbor structure features, protein neighbor structure feature, and protein next nearest neighbor structure features.

3. The method of claim 2 wherein each ensemble of shallow random networks is an ensemble of extreme learning machines (ELMs).

4. The method of claim 2, wherein each shallow random network consists of one hidden layer and one input layer where the weights are selected randomly.

5. The method of claim 2, wherein the first ensemble is a Loo ensemble and the at least one additional ensemble is a L_{0,1} ensemble.

6. The method of claim 2, wherein each node has global and local features computed once for each node and then used to create each edge sample for training and testing.

7. The method of claim 6, wherein the global features include node centrality.

8. The method of claim 6, wherein the local features include node degrees, entropy of its first order neighborhood, and random walk probability of its first-degree neighbors.

9. The method of claim 2, wherein each ensemble is trained independently of the others.

10. A system comprising:
a processor and a memory to execute computer program code to implement a method, including: accessing a training data set for a knowledge graph, the training data set including approved drug target interactions having drug, protein, and graph features;
generating a first ensemble of shallow random networks to contain drug node graph features, protein node graph features, drug structure features, and protein structure features for nodes directly connected to a candidate edge of the knowledge graph; and
generating at least one additional ensemble of shallow random networks to contain features from drug node neighbor graph features, protein node neighbor graph features, protein node nearest neighbor graph features, drug neighbor structure features, protein neighbor structure features, and protein next nearest neighbor structure features for neighboring nodes directly connected to a candidate edge of the knowledge graph and further contain protein structure features for next nearest neighbors;
for each ensemble, generating a prediction for the edge under consideration; and
averaging the predictions to generate an average prediction whether the candidate edge is a candidate for drug target interactions.

11. The system of claim 10, wherein each ensemble of shallow random networks is an ensemble of extreme learning machines (ELMs).

12. The system of claim 10, wherein each shallow random network consists of one hidden layer and one input layer where the weights are selected randomly.

13. The system of claim 10, wherein the first ensemble is a Loo ensemble and the at least one additional ensemble is a 0, 1 ensemble.

14. The system of claim 10, wherein each node has global and local features computed once for each node and then used to create each edge sample for training and testing.

15. The system of claim 14, wherein the global features include node centrality.

16. The system of claim 14, wherein the local features include node degrees, entropy of its first order neighborhood, and random walk probability of its first-degree neighbors.

17. The system of claim 10, wherein each ensemble is trained independently of the others;
assigning a class-based weight per each predicted class to each model in the ensemble based on results of the validation test to form a weighted output of the ensemble with a set of dense class-based weights.

18. A method of predicting a candidate edge of a graph neural network, comprising:
accessing a training data set and a validation data set for a knowledge graph, the training data set including interactions of a set of structural features and graph features;
generating a first ensemble of shallow random networks to contain left node graph features, right node graph features, left node structure features, and right node structure features for neighboring nodes directly connected to a candidate edge of the knowledge graph; and
generating at least one additional ensemble of shallow random networks for nodes that include at least some features contributed from next nearest neighbors;
for each ensemble, generating a prediction for the edge under consideration; and averaging the predictions to generate an average prediction whether the candidate edge is a candidate for drug target interactions.

19. The method of claim 18, wherein the at least one additional ensemble contains features from left node neighbor graph features, right node neighbor graph features, right next nearest neighbor graph features, left neighbor structure features, right neighbor structure features, and right next nearest neighbor structure features.
